(19) United States
(12) Patent Application Publication
(10) Pub. No.: US 2016/0264550 A2
(43) Pub. Date: Sep. 15, 2016

(54) PROCESS FOR ACYLATING AMINES

(71) Applicant: EVONIK TECHNOCHEMIE GMBH, DOSSENHEIM (DE)

(72) Inventors: Martin Bindl, Ludwigshafen (DE); Roland Herrmann, Maintal (DE); Gunter Knaup, Bruchkobel (DE)

(21) Appl. No.: 14/439,336

(22) PCT Filed: Oct. 4, 2013
(86) PCT No.: PCT/EP2013/070722

§ 371 Date: Apr. 29, 2015

Related U.S. Application Data
(60) Provisional application No. 61/722,004, filed on Nov. 2, 2012.

(51) Int. Cl. C07D 401/12

(52) U.S. Cl. CPC C07D 401/12

(57) ABSTRACT

The present invention is concerned with the formation of amide bonds. More specifically, the present invention, relates to processes for the manufacture of organogellant compounds (OG) as depicted below wherein L is selected from C₂-C₂₀ alkyl, C₆-C₂₀ aryl, C₇-C₂₀ alkylaryl, R¹ are side chain substituents and one of X¹, X² is nitrogen and the other two are carbon. Processes of the present invention employ reaction mixtures with beneficial flow characteristics allowing sufficient agitation of all parts of these reaction mixtures and thus achieving adequate mixing of reaction partners and/or dissipation of heat. The beneficial flow characteristics are achieved by using suitable activation for coupling the terminal pyridine-carboxylic acid.

The present invention is concerned with the formation of amide bonds. More specifically, the present invention, relates to processes for the manufacture of organogellant compounds (OG) as depicted below

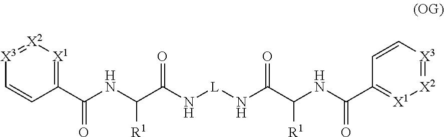

wherein L is a linking moiety of molecular weight from 14 g/mol to 500 g/mol, R¹ are sidechain substituents and X¹, X² and X³ are selected from carbon and nitrogen.

Organogellant compounds as depicted above are known in the art to serve as gellants to thicken liquid compositions. Such gellants have, for example, been described in WO 2011/112912 A1 and WO 2011/112887 A1.

Organogellant compounds also termed organogellants herein, in general are used to provide structure and a pleasant texture to liquid consumer products such as, for example, liquid detergent compositions. Furthermore, organogellants can be used to stabilize other components within such compositions such as, for example, enzymes and bleaches. However, organogellants need to be selected carefully for their respective application in order to prevent incompatibilities between organogellant and other components as well as unwanted side effects such as clouding.

Organogellants of the present invention offer significant advantages over other gellants currently in use, such as being compatible with a broad range of consumer products as well as not affecting product clarity.

Synthetic access to organogellants is described in WO 2011/112887 A1. However, the syntheses described therein are expensive and time-consuming. Accordingly, there is a need in the field for cheaper and faster access to organogellants.

Processes for the manufacture of organogellants are complicated by the fact that the presence of organogellants obtained alters crucial flow characteristics of the reaction mixture. As a result it may become impossible to provide sufficient agitation to all parts of the reaction mixture and thus achieving adequate mixing of reaction partners and/or dissipation of heat.

As presented herein, it was now found that the choice of activating agent used for coupling the terminal pyridine-carboxylic acid to the central diamide-linker has significant influence on the flow characteristics of the corresponding reaction mixture.

More specifically, it was found that activating the terminal pyridine-carboxylic acid with a sulfonyl chloride allowed reactions to proceed smoothly and with good yields, while activation with conventional acid chlorides resulted in reaction mixtures that could not be agitated as a result of their high viscosity thus producing very low yields.

Activation with sulfonyl chlorides allows pyridine-carboxylic acids to be coupled efficiently to the central diamide-linker of compounds OG presented above, thereby enabling access to organogellants.

Accordingly, the present invention provides processes for the manufacture of compounds of formula I

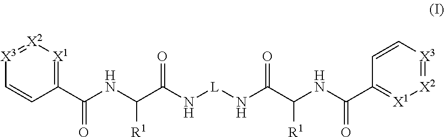

wherein in step (a) a compound of formula II

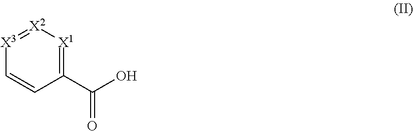

is reacted with a sulfonyl chloride RSC in solvent SA, with a base BA, at a temperature ⊖A, resulting in reaction mixture RA,
and wherein in step (b) a compound of formula III

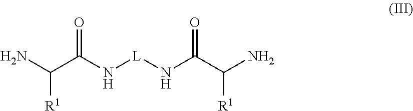

is dissolved in solvent SB with a base BB at a temperature ⊖B, resulting in reaction mixture RB subsequently;
and wherein in step (c) reaction mixtures RA and RB are combined at a temperature ⊖C, resulting in reaction mixture RC, wherein Σconc, representing the aggregated concentration of the compounds of formula II and III in reaction mixture RC, is in the range of CRL to CRU,
wherein
R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
and wherein one of X¹, X², X³ is nitrogen and the other two are carbon.

The following definitions are used in the context of the present invention:

An alkyl is a linear, branched, or cyclic hydrocarbon chain. It may also be a combination of linear, branched, and cyclic hydrocarbon chains. A Cₙ-Cₘ alkyl is an alkyl having n to m carbon atoms.

An aryl is an aromatic hydrocarbon. An aryl may be monocyclic or polycyclic. In the case of polycyclic aryls, the individual aromatic rings may be fused or may be connected by single carbon-carbon bonds. Examples of suitable aryls are phenyl, biphenyl, naphtyl, anthryl, or phenanthryl. A Cₙ,-Cₘ aryl is an aryl having n to m carbon atoms.

A heteroaryl is an aromatic hydrocarbon that contains 1 to 4 heteroatoms, preferably 1 to 2 heteroatoms. Heteroatoms are independently selected from nitrogen, oxygen, sulfur. A heteroaryl may be monocyclic or polycyclic. A heteroaryl may be attached to the main molecule through any of its carbon or nitrogen atoms. A Cₙ-Cₘ heteroaryl is a heteroaryl having n to m carbon atoms and 1 to 4 heteroatoms.

An alkylaryl is an aryl that is substituted with one or more alkyls. An alkylaryl may be attached to the remainder of the molecule through any of its alkyl or aryl carbon atoms. A Cₙ-Cₘ alkylaryl contains n to m carbon atoms.

An alkylheteroaryl is a heteroaryl that is substituted with one or more alkyls. The alkyl substituents may be attached to the heteroaryl through any of the carbon- or heteroatoms of the heteroaryl. The alkylheteroaryl group may be attached to the remainder of the molecule through any of the alkyl carbon atoms and/or the heteroaryl carbon- or heteroatoms.

A hydroxyalkyl is an alkyl carrying one or more hydroxyl groups. A Cₙ-Cₘ, hydroxyalkyl group contains n to m carbon atoms.

A thioether is a moiety wherein two alkyls are linked by a thioether bond. A Cₙ,-Cₘ, thioether group contains n to m carbon atoms in total. The thioether group may be attached to the remainder of the molecule through any of its carbon atoms.

An alkylhydroxyaryl is an alkylaryl, carrying hydroxyl groups on any of the aryl carbon atoms. The alkylhydroxyaryl group may be attached to the remainder of the molecule through any of its alkyl and/or aryl carbon atoms. A Cₙ-Cₘ alkylhydroxyaryl contains n to m carbon atoms.

An alkyl-C(O)Y is an alkyl carrying a C(O)Y-group, wherein C(O) is a carbonyl function and Y is selected from OR², —NH₂, —NHR³, —NHR³, —NR³R⁴; and wherein R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl. A Cₙ-Cₘ alkyl-C(O)Y contains n to m carbon atoms within the carbonyl-bound alkyl excluding the carbonyl carbon atom itself.

Bases BA and BB can be any bases including mixtures of bases suitable to perform the process of the present invention. Suitable organic bases act as proton acceptors and usually contain nitrogen atoms. In addition, suitable inorganic bases can be selected by a person of skill in the art.

Solvents SA and SB are aprotic organic solvents.

Suitable aprotic organic solvents comprise dichloromethane, methyl tert-butyl ether, tetrahydrofuran, acetonitrile, 1,4-dioxane, ethylene glycol dimethyl ether, methyl isobutyl ketone, methyl ethyl ketone, acetone, ethyl acetate, iso-propyl acetate, tert-butanol, 2-propanol or mixtures thereof. In the context of the present invention tert-Butanol and 2-propanol are considered as aprotic organic solvents.

Temperatures ⊖A, ⊖B, ⊖C can be selected in a range that is suitable to perform the corresponding reaction. Temperature ⊖A should be selected in a range where solvent SA is in the liquid state. Temperature ⊖B should be selected in a range where solvent SB is in the liquid state. Temperature ⊖C should be selected in a range where SA as well as SB is in the liquid state.

Σconc is the aggregated concentration of the compounds of formula II and III in reaction mixture RC. Σconc is defined by the term: Σconc=(amount of substance of compounds according to formula II in RC+amount of substance of compounds according to formula III in RC)/volume of reaction mixture RC. According to the present invention Σconc is selected in the range CRL to CRU.

CRL is the lower limit of Σconc and CRU is the upper limit of Σconc.

CRL can be selected as the lowest concentration that allows performing the process of the present invention with practically useful yields in practically useful periods of time. In preferred embodiments of the present invention CRL is selected from one of the following concentrations, however with the proviso as stated above, that this concentration allows performing the process of the present invention with practically useful yields in practically useful periods of time: 0.01M, 0.05M, 0.1M, 0.2M, 0.3M (with M denoting mol/l).

CRU can be selected as the highest concentration that allows dissolving the compounds of formulae land II in the solvent or solvent mixture that constitutes the basis of reaction mixture RC. In preferred embodiments of the present invention CRU is selected from one of the following concentrations, however with the proviso as stated above, that this concentration allows dissolving the compounds of formulae II and III in the solvent or solvent mixture that constitutes the basis of reaction mixture RC: 3.0M, 2.0M, 1.0M, 0.9M, 0.8M, 0.7M, 0.6M, 0.5M, 0.4M (with M denoting mol/l).

In preferred embodiments of the present invention CRL and CRU are selected from the following concentrations, however with the provisos as stated above, that concentration CRL allows performing the process of the present invention with practically useful yields in practically useful periods of time and that concentration CRU allows dissolving the compounds of formulae II and III in the solvent or solvent mixture that constitutes the basis of reaction mixture RC (with M denoting mol/l):

CRL=0.01M and CRU=3.0M, CRL=0.01M and CRU=2.0M, CRL=0.01M and CRU=1.0M, CRL=0.01 M and CRU=0.9M, CRL=0.01M and CRU=0.8M, CRL=0.01M and CRU=0.7M, CRL=0.01M and CRU=0.6M, CRL=0.01M and CRU=0.5M, CRL=0.01M and CRU=0.4M,

CRL=0.05M and CRU=3.0M, CRL=0.05M and CRU=2.0M, CRL=0.05M and CRU=1.0M, CRL=0.05M and CRU=0.9M, CRL=0.05M and CRU=0.8M, CRL=0.05M and CRU=0.7M, CRL=0.05M and CRU=0.6M, CRL=0.05M and CRU=0.5M, CRL=0.05M and CRU=0.4M

CRL=0.1M and CRU=3.0M, CRL=0.1M and CRU=2.0M, CRL=0.1M and CRU=1.0M, CRL=0.1M and CRU=0.9M, CRL=0.1M and CRU=0.8M, CRL=0.1M and CRU=0.7M, CRL=0.1M and CRU=0.6M, CRL=0.1M and CRU=0.5M, CRL=0.1M and CRU=0.4M

CRL=0.2M and CRU=3.0M, CRL=0.2M and CRU=2.0M, CRL=0.2M and CRU=1.0M, CRL=0.2M and CRU=0.9M, CRL=0.2M and CRU=0.8M, CRL=0.2M and CRU=0.7M, CRL=0.2M and CRU=0.6M, CRL=0.2M and CRU=0.5M, CRL=0.2M and CRU=0.4M

CRL=0.3M and CRU=3.0M, CRL=0.3M and CRU=2.0M, CRL=0.3M and CRU=1.0M, CRL=0.3M and CRU=0.9M, CRL=0.3M and CRU=0.8M, CRL=0.3M and CRU=0.7M, CRL=0.3M and CRU=0.6M, CRL=0.3M and CRU=0.5M, CRL=0.3M and CRU=0.4M

The stoichiometric relation between compounds according to formula II and compounds according to formula III in reaction mixture RC is selected in a range suitable to perform the process of the present invention. A suitable stoichiometric relation between compounds according to formula II and compounds according to formula III in reaction mixture RC can be selected in the range: (amount of substance of compounds according to formula II in RC)/(amount of substance of compounds according to formula III in RC)=1.8:1 to 4:1. In a preferred embodiment of the present invention the stoichiometric relation between compounds according to formula II and compounds according to formula III in reaction mixture RC is selected in the range: (amount of substance of compounds according to formula II in RC)/(amount of substance of compounds according to formula III in RC)=1.8:1 to 2.6:1.

Sulfonyl chlorides can be any sulfonyl chloride compounds (R—SO₂—Cl) suitable to perform the process of the present invention

Suitable sulfonyl chlorides comprise p-toluenesulfonyl chloride, p-bromobenzenesulfonyl chloride, p-n itrobenzenesulfonyl chloride, methanesulfonyl chloride.

Compounds according to formula II are commercially available. Compounds according to formula III can be accessed from commercially available diamines and amino acids via standard peptide chemistry well known to a person of skill in the art.

In a preferred embodiment of the present invention R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine, tyrosine, tryptophan, serine, threonine, glutamine, asparagine.

In another preferred embodiment of the present invention X¹ and X² are carbon and X³ is nitrogen. In another preferred embodiment of the present invention X¹ and X³ are carbon and X² is nitrogen. In another preferred embodiment of the present invention X² and X³ are carbon and X¹ is nitrogen.

In another preferred embodiment of the present invention L is selected from a C₆-C₁₂ linear alkyl group, a 1,4-dimethylcyclohexyl group and a xylene group.

In another preferred embodiment of the present invention solvents SA and SB are selected from dichloromethane, methyl-isobutyl ketone, acetonitrile and mixtures thereof.

In another preferred embodiment of the present invention bases BA and BB are selected from triethylamine, di-isopropyl-ethylamine,1,5-Diazabicyclo[4.3.0]non-5-ene, 1,4-Diazabicyclo[2.2.2]octane, 1,8-Diazabicyclo[5.4.0]undec-7-ene and mixtures thereof. In another preferred embodiment of the present invention bases BA and BB are selected from triethylamine, di-isopropyl-ethylamine and mixtures thereof.

In another preferred embodiment of the present invention temperatures ⊖A, ⊖B and ⊖C are selected in the interval from 0° C. to 30° C. In another preferred embodiment of the present invention temperatures ⊖A, ⊖B are selected in the interval from 0° C. to 30° C., and temperature ⊖C is selected in the interval from 0° C. to 10° C.

In another preferred embodiment of the present invention CRL is selected as 0.1 mol/l and CRU is selected as 0.6 mol/l.

In another preferred embodiment of the present invention the sulfonyl chloride RSC is selected from p-toluenesulfonyl chloride, methanesulfonyl chloride.

In another preferred embodiment of the present invention R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A, ⊖B and ⊖C are selected in the interval from 0° C. to 30° C.;
concentrations CRL and CRU are selected as CRL=0.1 mol/l, CRU=0.6 mol/l;
the sulfonyl chloride RSC is selected from p-toluenesulfonyl chloride, methanesulfonyl chloride.

In another preferred embodiment of the present invention R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine, tyrosine, tryptophan, serine, threonine, glutamine, asparagine;
L is selected from a C₆-C₁₂ linear alkyl group, a 1,4-dimethylcyclohexyl group and a xylene group;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is selected from p-toluenesulfonyl chloride, methanesulfonyl chloride.

In another preferred embodiment of the present invention R¹ is independently selected from a hydrogen atom an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine;
L is selected from a C₆-C₁₂ linear alkyl group, a 1,4-dimethylcyclohexyl group and a xylene group;
Y is independently selected from OR², NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
X¹ and X² are carbon and X³ is nitrogen;
solvents SA and SB are selected from dichloromethane, methyl-isobutyl ketone, acetonitrile and mixtures thereof;
bases BA and BB are selected as triethylamine;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is selected from p-toluenesulfonyl chloride, methanesulfonyl chloride.

In another preferred embodiment of the present invention R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A, ⊖B and ⊖C are selected in the interval from 0° C. to 30° C.; concentrations CRL and CRU are selected as CRL=0.1 mol/l, CRU=0.6 mol/l;
the sulfonyl chloride RSC is methanesulfonyl chloride.

In another preferred embodiment of the present invention R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine, tyrosine, tryptophan, serine, threonine, glutamine, asparagine;
L is selected from a C₆-C₁₂ linear alkyl group, a 1,4-dimethylcyclohexyl group and a xylene group;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is methanesulfonyl chloride.

In another preferred embodiment of the present invention R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine;
L is selected from a C₆-C₁₂ linear alkyl group, a 1,4-dimethylcyclohexyl group and a xylene group;
Y is independently selected from OR², NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
X¹ and X² are carbon and X³ is nitrogen;
solvents SA and SB are selected from dichloromethane, methyl-isobutyl ketone, acetonitrile and mixtures thereof;
bases BA and BB are selected as triethylamine;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l; the sulfonyl chloride RSC is methanesulfonyl chloride.

### EXAMPLES

### Example 1

### Coupling of Isonicotinic Acid to Valine Methyl Ester

Method A: Coupling Via MsCl

### Example 1 a:

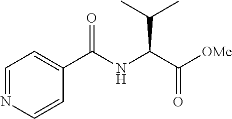 [SMILES: COC(=O)[C@@H](NC(=O)c1ccncc1)C(C)C]

Batch 1:

Isonicotinic acid (15.3 g, 0.13 mol) is suspended in acetonitrile (250 mL) and triethylamine (18.0 mL, 0.13 mol) is added. The reaction mixture is cooled to 0-5° C. and methanesulfonyl chloride (10.1 mL, 0.13 mol) (MsCl) is added. The mixture is stirred 15 min at 15° C. and recooled to 0-5° C.

Batch 2:

Valine ethyl ester HCl (19 g, 0.11 mol) and triethylamine (58.1 mL, 0.42 mol) are dissolved in acetonitrile (164 mL) at room temperature.

The mixture of batch 2 is added to the activated isonicotinic acid solution (batch 1) during 1 h at 0-5° C. The reaction mixture is stirred for 1 h at room temperature. Water (80 mL) is added and acetonitrile is removed under vacuum (50 mbar). Methyl isobutyl ketone (130 mL) is added to the residue and the pH=8.5 is adjusted by the addition of NaOH (50%) solution. The aqueous phase is separated and washed three times with methyl isobutyl ketone (3×30 mL). For the washings, the pH of the aqueous phase is increased to 10-11. The organic layers are combined and the solvent is evaporated. The red-brown, crystalline solid is dried at 50 ° C. under vacuum. Yield: 24.4 g (91%).

### Example 1 b:

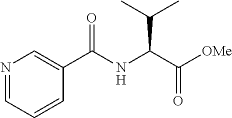 [SMILES: COC(=O)[C@@H](NC(=O)c1cccnc1)C(C)C]

The compound is prepared in the analogue manner as described above.

Method A: Yield: 85%; Method B: Yield: 81%

¹H-NMR (600 MHz, CDCl₃): δ=9.05 (t, J=2.4 Hz, 1H), 8.75 (d, J=4.8 Hz, 1H), 8.14 (m, 1H), 7.41 (m, 1H), 6.75 (d, J=6.0 Hz, 1H), 4.79 (t, J=7.8 Hz, 1H), 3.79 (s, 3H), 2.32-2.29 (m, 1H), 1.03 (d, J=6.6 Hz, 3H), 1.01 (d, J=7.2 Hz, 3H) ppm.

### Example 1c:

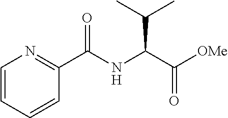 [SMILES: COC(=O)[C@@H](NC(=O)c1ccccn1)C(C)C]

The compound is prepared in the analogue manner as described above.

Method A: Yield: 89%; Method B: Yield: 95%

¹H-NMR (600 MHz, CDCl₃): δ=8.60 (m, 1H), 8.53 (d, J=6.0 Hz, 1H), 8.18 (d, J=12.0 Hz, 1H), 7.85 (m, 1H), 7.44 (m, 1H), 4.74 (t, J=7.8 Hz, 1H), 3.77 (s, 3H), 2.34-2.30 (m, 1H), 1.04 (d, J=6.6 Hz, 3H), 1.02 (d, J=7.2 Hz, 3H) ppm.

Method B: Coupling Via Acid Chloride

### Example 1d:

Valine ethyl ester HCl (19 g, 0.11 mol) is suspended in acetonitrile (164 mL) and cooled to 10° C. Isonicotinic acid chloride HCl (28.2 g, 0.16 mol) is added to the mixture. Triethylamine (42.4 g, 0.42 mol) is added drop wise at 5-10° C. during 2.5 h. The red-brown solution is stirred at room temperature for 45 min and treated with water (50 mL) after complete conversion. Acetonitrile is evaporated under vacuum (50 mbar). Methyl isobutyl ketone (95 mL) is added to the aqueous phase and the pH is adjusted to 8.5 by addition of NaOH solution (50% w/w %). Additional water (32 mL) and methyl isobutyl ketone (32 mL) are added, the aqueous phase is separated and extracted with methyl isobutyl ketone (32 mL). The combined organic layers are washed three times with water (3×32 mL). For the washings, the pH of the aqueous phase is adjusted to 10. The solvent of the organic phase is evaporated and the red-brown, crystalline solid is dried at 50° C. under vacuum. Yield: 24.0 g (90%).

¹H-NMR (600 MHz, DMSO): δ=8.92 (d, J=7.8 Hz, 1H), 8.74 (d, J=4.8 Hz, 2H), 7.78 (d, J=4.2 Hz, 2H), 4.32 (t, J=7.8 Hz, 1H), 3.67 (s, 3H), 2.21-2.17 (m, 1H), 0.98 (d, J=6.6 Hz, 3H), 0.94 (d, J=7.2 Hz, 3H) ppm.

### Example 2

### coupling of isonicotinic acid to N,N-Bis-L-valoyl-1,12-diaminododecane

Method C: Coupling Via MsCl

### Example 2a:

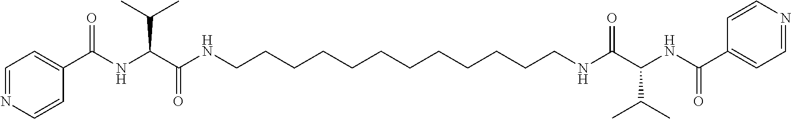 [SMILES: CC(C)[C@H](NC(=O)c1ccncc1)C(=O)NCCCCCCCCCCCCNC(=O)[C@H](NC(=O)c1ccncc1)C(C)C]

Batch 1:

Isonicotinic acid (5.4 g, 44 mmol) is suspended in methyl isobutyl ketone (100 mL) and triethylamine (6.08 mL, 44 mmol) is added. The reaction mixture is cooled to 0-5° C. and methanesulfonyl chloride (3.4 mL, 44 mmol) is added. The mixture is stirred 15 min at 15° C. and recooled to 0-5° C.

Batch 2:

N,N-Bis-L-valoyl-1,12-diaminododecane (8.0 g, 20 mmol) and triethylamine (7.0 mL, 50 mmol) are dissolved in dichloromethane (60 mL) at room temperature.

The mixture of batch 2 is added to the activated isonicotinic acid solution (batch 1) during 1 h at 0-5° C. The reaction mixture is stirred for 1 h at room temperature. 60 mL solvent are distilled off and the residue is treated with methyl isobutyl ketone (100 mL). Further 60 mL solvent are distilled off at 700-800 mbar. The residue is treated with additional methyl isobutyl ketone (20 mL) and water (60 mL). The pH is adjusted to 11 by addition of NaOH (50 w/w % solution). The organic phase is separated at 70° C. and washed with water (60 mL) at 70° C. Heptane (120 mL) is added to the organic phase, which is subsequently heated to reflux. The solution is cooled to 65° C., seeded and stirred at 65° C. for 30 min. The resulting suspension is cooled to room temperature during 1 h and stirred an additional hour at this temperature. The product is isolated by filtration and washing with methyl isobutyl ketone (2×40 mL). The white solid is dried at 50° C. under vacuum. Yield: 10.0 g (82%).

### Example 2b:

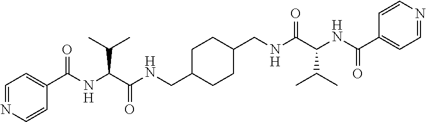 [SMILES: CC(C)[C@H](NC(=O)c1ccncc1)C(=O)NCC1CCC(CNC(=O)[C@H](NC(=O)c2ccncc2)C(C)C)CC1]

The compound is prepared in the analogue manner to method C. Yield: 73%.

¹H-NMR (600 MHz, DMSO/HCl): δ=9.32 (d, J=8.4 Hz, 2H), 9.11 (d, J=5.4 Hz, 4H), 8.48 (d, J=4.8 Hz, 4H), 8.36 (s, 2H), 4.29 (t, J=7.2 Hz, 2H), 3.09-2.84 (m, 4H), 2.21-2.17 (m, 2H), 1.71-1.61 (m, 3H), 1.39-1.30 (m, 5H), 0.95 (d, J=6.6 Hz, 6H), 0.94 (d, J=6.6 Hz, 6H), 0,88 (s, 2H) ppm.

### Example 2c:

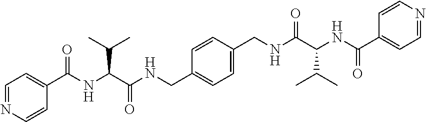 [SMILES: CC(C)[C@H](NC(=O)c1ccncc1)C(=O)NCc1ccc(CNC(=O)[C@H](NC(=O)c2ccncc2)C(C)C)cc1]

The compound is prepared in the analogue manner to method C. Yield: 57%.

¹H-NMR (600 MHz, DMSO/HCl): δ=9.31 (d, J=8.4 Hz, 2H), 9.11 (d, J=6.6 Hz, 4H), 8.91 (t, J=6.0 Hz, 2H), 8.49 (d, J=6.6 Hz, 4H), 7.23 (s, 4H), 4.34-4.29 (m, 6H), 2.26-2.20 (m, 2H), 0.96 (d, J=7.2 Hz, 6H), 0.92 (d, J=6.6 Hz, 6H) ppm.

### Example 2d:

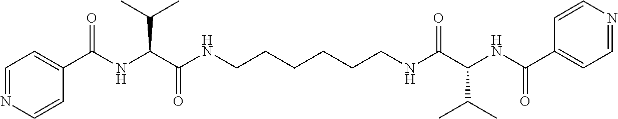 [SMILES: CC(C)[C@H](NC(=O)c1ccncc1)C(=O)NCCCCCCNC(=O)[C@H](NC(=O)c1ccncc1)C(C)C]

The compound is prepared in the analogue manner to method C. Yield: 64%.

¹H-NMR (600 MHz, DMSO/HCl): δ=9.34 (d, J=8.4 Hz, 2H), 9.09 (d, J=5.4 Hz, 4H), 8.47 (d, J=5.4 Hz, 4H), 8.33 (t, J=6.0 Hz, 2H), 4.26 (t, J=8.4 Hz, 2H), 3.11-3.08 (m, 2H), 3.03-3.01 (m, 2H), 2.19-2.15 (m, 2H), 1.39 (s, 4H), 1.26 (s, 4H), 0.94 (d, J=6.6 Hz, 6H), 0.92 (d, J=6.6 Hz, 6H) ppm.

Method D: Coupling Via Acid Chloride:

### Example 2e:

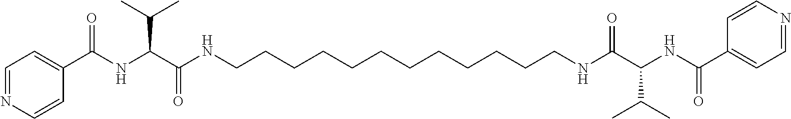 [SMILES: CC(C)[C@H](NC(=O)c1ccncc1)C(=O)NCCCCCCCCCCCCNC(=O)[C@H](NC(=O)c1ccncc1)C(C)C]

Test 1:

N,N-Bis-L-valoyl-1,12-diaminododecane (4.0 g, 10 mmol) is suspended in methyl isobutyl ketone (50 mL) and dichloromethane (30 mL) and cooled to 0° C. Isonicotinic acid chloride HCl (3.92 g, 22 mmol) is added to the mixture. Triethylamine (9 mL, 65 mmol) is added drop wise at 5-10° C. during 2.5 h. After dosing triethylamine, the mixture reached a level of viscosity, which made stirring (with sealed precision glass (KPG) stirrer) impossible.

The reaction mixture is treated with water (100 mL) and dichloromethane and methyl isobutyl ketone are evaporated under vacuum (50 mbar). Methyl isobutyl ketone (150 mL) is added to the aqueous phase and the pH is adjusted to 8.5 by addition of NaOH solution (50% w/w %). Additional water (100 mL) and methyl isobutyl ketone (50 mL) are added, the aqueous phase is separated and extracted with methyl isobutyl ketone (50 mL). The combined organic layers are washed three times with water (3×50 mL). For the washings, the pH of the aqueous phase is adjusted to 10. The solvent of the organic phase is evaporated and the white solid is dried at 50° C. under vacuum Yield: 0.48 g (7%).

Test 2:

N,N-Bis-L-valoyl-1,12-diaminododecane (4.0 g, 10 mmol) is suspended in dichloromethane (80 mL) and cooled to 0° C. Isonicotinic acid chloride HCl (3.92 g, 22 mmol) is added to the mixture. Triethylamine (9 mL, 65 mmol) is added drop wise at 5-10° C. during 2.5 h. After dosing triethylamine, the mixture reached a level of viscosity, which made stirring (with sealed precision glass (KPG) stirrer) impossible. The reaction mixture is treated with water (100 mL) and dichloromethane is evaporated under vacuum (50 mbar). Methyl isobutyl ketone (150 mL) is added to the aqueous phase and the pH is adjusted to 8.5 by addition of NaOH solution (50% w/w %). Additional water (100 mL) and methyl isobutyl ketone (50 mL) are added, the aqueous phase is separated and extracted with methyl isobutyl ketone (50 mL). The combined organic layers are washed three times with water (3×50 mL). For the washings, the pH of the aqueous phase is adjusted to 10. The solvent of the organic phase is evaporated and the white solid is dried at 50° C. under vacuum Yield: 0.32 g (5%).

Test 3:

N,N-Bis-L-valoyl-1,12-diaminododecane (4.0 g, 10 mmol) is suspended in acetonitrile (100 mL) and cooled to 0° C. Isonicotinic acid chloride HCl (5.0 g, 28 mmol) is added to the mixture. Triethylamine (9 mL, 65 mmol) is added drop wise at 5-10° C. during 2.5 h. After 1.5 h of dosing triethylamine, the mixture reached a level of viscosity, which made stirring (with sealed precision glass (KPG) stirrer) impossible.

The reaction mixture is treated with water (100 mL) and acetonitrile is evaporated under vacuum (50 mbar). Methyl isobutyl ketone (150 mL) is added to the aqueous phase and the pH is adjusted to 8.5 by addition of NaOH solution (50% w/w %). Additional water (100 mL) and methyl isobutyl ketone (50 mL) are added, the aqueous phase is separated and extracted with methyl isobutyl ketone (50 mL). The combined organic layers are washed three times with water (3×50 mL). For the washings, the pH of the aqueous phase is adjusted to 10. The solvent of the organic phase is evaporated and the white solid is dried at 50° C. under vacuum Yield: 0.38 g (6%).

¹H-NMR (600 MHz, DMSO): δ=8.74 (dd, J=4.2, 1.8 Hz, 4H), 8.63 (d, J=8.4 Hz, 2H), 8.06 (t, J=5.4 Hz, 2H), 7.84 (dd, J=4.5, 1.8 Hz, 4H), 4.25 (t, J=8.4 Hz, 2H), 3.16-3.12 (m, 2H), 3.11-2.98 (m, 2H), 2.14-2.07 (m, 2H), 1.40 (s, 4H), 1.22-2.18 (m, 16H), 0.92 (d, J=6.6 Hz, 6H), 0.91 (d, J=6.6 Hz, 6H) ppm.

1. Process for the manufacture of compounds according to formula I, 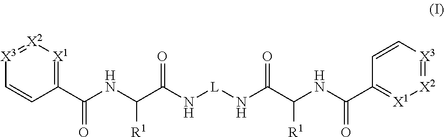
wherein in step (a) a compound of formula II 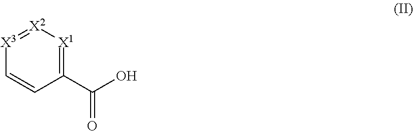
is reacted with a sulfonyl chloride RSC in solvent SA, with a base BA, at a temperature ⊖A, resulting in reaction mixture RA,
and wherein in step (b) a compound of formula III 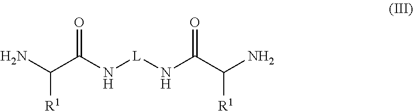
is dissolved in solvent SB with a base BB at a temperature ⊖B, resulting in reaction mixture RB subsequently;
and wherein in step (c) reaction mixtures RA and RB are combined at a temperature ⊖C, resulting in reaction mixture RC, wherein Σconc, representing the aggregated concentration of the compounds of formula II and III in reaction mixture RC, is in the range of CRL to CRU,
wherein
R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
and wherein one of X¹, X², X³ is nitrogen and the other two are carbon.

2. Process according to claim 1, wherein R¹ is independently selected from a hydrogen atom, an n-butyl group, a t-butyl group, a propyl group, a cyclopropyl group, an ethyl group, a C1-C4 alkyl-C(O)Y and one of the side chains of amino acids alanine, valine, leucine, isoleucine, methionine, phenylalanine, tyrosine, tryptophan, serine, threonine, glutamine, asparagine.

3. Process according to claim 1, wherein L is selected from a C₆-C₁₂ linear alkyl group, a 1,4-dimethylcyclohexyl group and a xylene group.

4. Process according to claim 1, wherein solvents SA and SB are selected from dichloromethane, methyl-isobutyl ketone, acetonitrile and mixtures thereof.

5. Process according to claim 1, wherein bases BA and BB are selected from triethylamine, di-isopropyl-ethylamine, 1,5-Diazabicyclo[4.3.0]non-5-ene, 1,4-Diazabicyclo[2.2.2]octane, 1,8-Diazabicyclo[5.4.0]undec-7-ene and mixtures thereof.

6. Process according to claim 1, wherein temperatures ⊖A, ⊖B and ⊖C are selected in the interval from 0° C. to 30° C.

7. Process according to claim 1, wherein temperatures ⊖A, ⊖B are selected in the interval from 0° C. to 30° C., and wherein temperature ⊖C is selected in the interval from 0° C. to 10° C.

8. Process according to claim 1, wherein CRL is selected as 0.1 mol/l and CRU is selected as 0.6 mol/l.

9. Process according to claim 1, wherein the sulfonyl chloride RSC is selected from p-toluenesulfonyl chloride, methanesulfonyl chloride.

10. Process according to claim 1, wherein
R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A, ⊖B and ⊖C are selected in the interval from 0° C. to 30° C.;
concentrations CRL and CRU are selected as CRL=0.1 mol/l, CRU=0.6 mol/l;
the sulfonyl chloride RSC is selected from p-toluenesulfonyl chloride, methanesulfonyl chloride.

11. Process according to claim 1, wherein
R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine, tyrosine, tryptophan, serine, threonine, glutamine, asparagine;
L is selected from a C₆-C₁₂ linear alkyl group, a 1,4-dinnethylcyclohexyl group and a xylene group;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is selected from p-toluenesulfonyl chloride, methanesulfonyl chloride.

12. Process according to claim 1, wherein
R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine;
L is selected from a C₆-C₁₂ linear alkyl group, a 1,4-dimethylcyclohexyl group and a xylene group;
Y is independently selected from OR², NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
X¹ and X² are carbon and X³ is nitrogen;
solvents SA and SB are selected from dichloromethane, methyl-isobutyl ketone, acetonitrile and mixtures thereof;
bases BA and BB are selected as triethylamine;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is selected from p-toluenesulfonyl chloride, methanesulfonyl chloride.

13. Process according to claim 1, wherein
R¹ is independently selected from hydrogen atom, C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl, C₁-C₄ alkyl-C(O)Y;
L is selected from C₂-C₂₀ alkyl, C₆-C₂₀ aryl, C₇-C₂₀ alkylaryl;
Y is independently selected from OR², —NH₂, —NHR³, —NR³R⁴;
R², R³ R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A, ⊖B and ⊖C are selected in the interval from 0° C. to 30° C.;
concentrations CRL and CRU are selected as CRL=0.1 mol/l, CRU=0.6 mol/l;
the sulfonyl chloride RSC is methanesulfonyl chloride.

14. Process according to claim 1, wherein
R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine, tyrosine, tryptophan, serine, threonine, glutamine, asparagine;
L is selected from a C₆-C₁₂ linear alkyl group, a 1,4-dimethylcyclohexyl group and a xylene group;
Y is independently selected from OR², —NH₂, —NHR³, —NR³R⁴;
R², R³ and R⁴ are independently selected from C₁-C₄ alkyl, C₁-C₄ hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
one of X¹, X², X³ is nitrogen and the other two are carbon;
solvents SA and SB are selected from dichloromethane, methyl-isobutyl ketone, acetonitrile and mixtures thereof;
bases BA and BB are selected from triethylamine, di-isopropyl-ethylamine and mixtures thereof;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is methanesulfonyl chloride.

15. Process according to claim 1, wherein
R¹ is independently selected from a hydrogen atom, an n-butyl group, a t-butyl group, a propyl group, a cyclopropyl group, an ethyl group, a C₁-C₄ alkyl-C(O)Y and one of the side chains of amino acids alanine, valine, leucine, isoleucine, methionine, phenylalanine;
L is selected from a C₆-C₁₂ linear alkyl group, a 1,4-dinnethylcyclohexyl group and a xylene group;
Y is independently selected from OR², NH₂, —NHR³, —NR³R⁴;
R², R³ and R⁴ are independently selected from C₁-C₄ alkyl, C1-C4 hydroxyalkyl, C₁-C₄ thioether, C₆-C₂₀ aryl, C₇-C₂₀ alkylaryl, C₇-C₂₀ alkylhydroxyaryl, C₄-C₂₀ alkylheteroaryl;
X¹ and X² are carbon and X³ is nitrogen;
solvents SA and SB are selected from dichloromethane, methyl-isobutyl ketone, acetonitrile and mixtures thereof;
bases BA and BB are selected as triethylamine;
temperatures ⊖A and ⊖B are selected in the interval from 0° C. to 30° C., and temperature ⊖C is selected in the interval from 0° C. to 10° C.;
concentrations CRL and CRU are selected as CRL=0.2 mol/l, CRU=0.5 mol/l;
the sulfonyl chloride RSC is methanesulfonyl chloride.

16. Process according to claim 2, wherein L is selected from a C₆-C₁₂ linear alkyl group, a 1,4-dimethylcyclohexyl group and a xylene group.

17. Process according to claim 2, wherein solvents SA and SB are selected from dichloromethane, methyl-isobutyl ketone, acetonitrile and mixtures thereof.

18. Process according to claim 3, wherein solvents SA and SB are selected from dichloromethane, methyl-isobutyl ketone, acetonitrile and mixtures thereof.

19. Process according to claim 2, wherein bases BA and BB are selected from triethylamine, di-isopropyl-ethylamine, 1,5-Diazabicyclo[4.3.0]non-5-ene, 1,4-Diazabicyclo[2.2.2]octane, 1,8-Diazabicyclo[5.4.0]undec-7-ene and mixtures thereof.

20. Process according to claim 3, wherein bases BA and BB are selected from triethylamine, di-isopropyl-ethylamine, 1,5-Diazabicyclo[4.3.0]non-5-ene, 1,4-Diazabicyclo[2.2.2]octane, 1,8-Diazabicyclo[5.4.0]undec-7-ene and mixtures thereof.
